(19) United States
(12) Patent Application Publication
(10) Pub. No.: US 2011/0306871 A1
(43) Pub. Date: Dec. 15, 2011

(54) Multiple-People MRI Scanner That Can Simultaneously Scan More Than One Person

(75) Inventor: Ray F. Lee, Princeton, NJ (US)

(73) Assignee: Ray F. Lee, Princeton, NJ (US)

(21) Appl. No.: 12/608,978

(22) Filed: Oct. 29, 2009

Related U.S. Application Data
(60) Provisional application No. 61/109,292, filed on Oct. 29, 2008.

(51) Int. Cl. A61B 5/055

(52) U.S. Cl. 600/422

(57) ABSTRACT

A system and method perform magnetic resonance imaging (“MRI”) on more than one people simultaneously with one MRI scanner. The system may include a superconducting or non-superconducting magnet, a set of three dimensions of magnetic field gradients, RF coils, and magnetic field shimming coil set that can accommodate more than one people within one scanner. The system and method may be used for performing MRI on more than one interacting or non-interacting people for anatomic, functional, metabolic, and/or molecular imaging and studies.

### FIELD OF THE INVENTION

The present invention relates generally to Magnetic Resonance Imaging (“MRI”). More specifically, the present invention relates to systems and methods for performing MRI on more than one interacting or non-interacting people simultaneously within one MRI scanner for anatomic, functional, metabolic, and/or molecular imaging and studies.

### BACKGROUND INFORMATION

One of the major functions of the human brain is to mediate interactions with other people, such as inter-personal communication and physical contact. Understanding such dynamic interactions between two minds is essential for characterizing human social behavior.

Until recently, studying brain mechanisms underlying social interactions has not been possible due to the lack of measurable methods to observe two interacting minds simultaneously. However, some progress has been made during the past ten years [1]. For example, Montague and colleagues developed a technique known as “hyperscanning” that uses the interne to connect and synchronize two MRI scanners while subjects were scanned performing a simple deception game [2]. Similarly, Babiloni and colleagues studied dyads participating in the Prisoner's Dilemma paradigm while recording EEG signals [3].

In line with this research, we have developed a novel MRI head coil that allows for the acquisition of fMRI signals from at least two subjects' brains while the subjects are in close proximity to one another, allowing for social interactions not possible with the remote hyperscanning technique. Unlike hyperscanning, which scans two people in separate scanners with only a visual or audio connection between them, the proposed device allows for fMRI studies of two people within close physical contact.

### SUMMARY OF THE INVENTION

In view of the foregoing, one of the objects of the present invention is to provide systems and methods for performing MM on more than one people simultaneously within one MRI scanner.

It is another object of the present invention to provide systems and methods for design and manufacture a MRI scanner which includes at least a superconducting or non-superconducting magnet, a set of three dimensions of magnetic field gradients, RF coils, and magnetic field shimming coil set that can accommodate more than one people within one scanner.

It is another object of the present invention to provide systems and methods for performing MRI on more than one interacting or non-interacting people for anatomic, functional, metabolic, and/or molecular imaging and studies.

These and other objects of the present invention are accomplished using an exemplary embodiment which can include a twin-head coil comprising: two circular polarized volume coils side-by-side for two people; the two component coils are decoupled by both their geometrical orientation and decoupling interface circuit system [4].

### REFERENCES

[1] C. Frith and U. Frith, “Interacting Minds—A Biological Basis,” Science, vol. 286, pp. 1692-1695, November 1999.
[2] P. R. Montague, G. Berns, J. Cohen, S. McClure, G. Pagnoni, M. Dhamala, M. Wiest, I. Karpov, R. King, N. Apple, and R. Fisher, “Hyperscanning: Simulaneous fMRI during Linked Social Interactions,” NeuroImage, vol. 16, pp. 1159-1164, 2002.
[3] F. Babiloni, F. Cincotti, D. Mattia, F. de vico Fallani, A. Tocci, L. Bianchi, S. Salinari, M. Marchiani, A. Colosimo, and Astolfi, “High Resolution EEG Hyperscanning during a Card Game”, pp. 4957-4960, The 29^{th} Annual International Conference of IEEE EMBS 2007, Lyon, France
[4] R. F. Lee, R. Giaquinto, and C. Hardy, “Coupling and Decoupling Theory and its Application to the MRI Phased Array,” Magnetic Resonance in Medicine, vol. 48, pp. 203-213, 2002.

### BRIEF DESCRIPTION OF THE DRAWINGS

The foregoing and other objects of the present invention will be apparent upon consideration of the following detailed description, taken in conjunction with the accompanying drawings, in which like reference characters refer to like parts throughout, and in which:

FIG. 1 is a visual illustration of an exemplary embodiment of a elliptical superconducting magnet according to the present invention;

FIG. 2 is a visual picture of an exemplary embodiment of the twin-head coil according to the present invention;

FIG. 3 are visual diagram of exemplary layouts of the interface between the twin-head coil and MRI scanner in accordance with an exemplary embodiment of the present invention.

What is claimed is:

1. A method for simultaneously acquiring magnetic resonance signals from more than one people in one magnetic resonance imaging scanner, comprising the steps of:
a. Providing a MRI scanner whose dimensions and physical features of imaging components such as magnet, gradient, and RF coil can accommodate at least two people;
b. Such scanner can simultaneously excite the spins and simultaneously receive their magnetic resonance signals from at least two people;
c. The magnetic resonance images reconstructed from such scanner can reveal either interaction and/or non-interaction between the plurality of people inside the scanner.

2. A method for simultaneously acquiring magnetic resonance signals from two people in one magnetic resonance imaging scanner, comprising the steps of:
a. Providing a MRI scanner which has elliptical cylinder dimension inner bore can accommodate two people;
b. Such scanner can simultaneously excite the spins and simultaneously receive their magnetic resonance signals from two people;
c. The magnetic resonance images reconstructed from such scanner can reveal either interaction and/or non-interaction between two people inside the scanner.

3. The method of claim 2, wherein step a further includes the steps of:
a. Constructing a elliptical cylinder inner bore superconducting or non-superconducting magnet that can generate homogeneous static magnetic field whose volume can accommodate two people;
b. Constructing three direction magnetic field gradient coils which can produce linear magnetic gradient region that can accommodate two people.
c. Constructing RF coils that can transmit and/or receive MR signals in the region that can accommodate two people.
d. Constructing shimming coil set that can shim an elliptical volume that is large enough for accommodating two people.

4. The method of claim 2, wherein step b further includes the step of acquiring MR signal for anatomic MR imaging of two people inside scanner.

5. The method of claim 2, wherein step b further includes the step of acquiring MR signal for functional MR imaging of two people inside scanner.

6. The method of claim 2, wherein step b further includes the step of acquiring MR signal for molecular MR imaging of two people inside scanner.

7. The method of claim 2, wherein step b further includes the step of acquiring MR signal for metabolic imaging of two people inside scanner.

8. The method of claim 2, wherein step c further includes the step of reconstructing anatomic MR images of interaction and or non-interaction between two people inside scanner.

9. The method of claim 2, wherein step c further includes the step of reconstructing functional MR images of interaction and or non-interaction between two people inside scanner.

10. The method of claim 2, wherein step c further includes the step of reconstructing molecular MR images of interaction and or non-interaction between two people inside scanner.

11. The method of claim 2, wherein step c further includes the step of reconstructing metabolic MR images of interaction and or non-interaction between two people inside scanner.

12. The method of claim 3, wherein step c further includes the step of constructing two circular polarized volume coils side-by-side for two people. The two component coils are decoupled by both their geometrical orientation and decoupling interface circuit system.

13. The method of claim 8 further includes step of analyzing the physical sexual compatibility between man and woman.

14. The method of claim 9 further includes step of analyzing the psychological compatibility between man and woman.

15. Apparatus for simultaneously acquiring magnetic resonance signals from more than one people in one magnetic resonance imaging scanner, comprising:
a. An arbitrary shape of inner bore superconducting or non-superconducting magnet that can generate homogeneous static magnetic field whose volume can accommodate more than one people;
b. Three direction magnetic field gradient coils which can produce linear magnetic gradient region that can accommodate more than one people.
c. RF coils that can transmit and/or receive MR signals in the region that can accommodate more than one people.
d. Shimming coil set that can shim an elliptical volume that is large enough for accommodating more than one people.

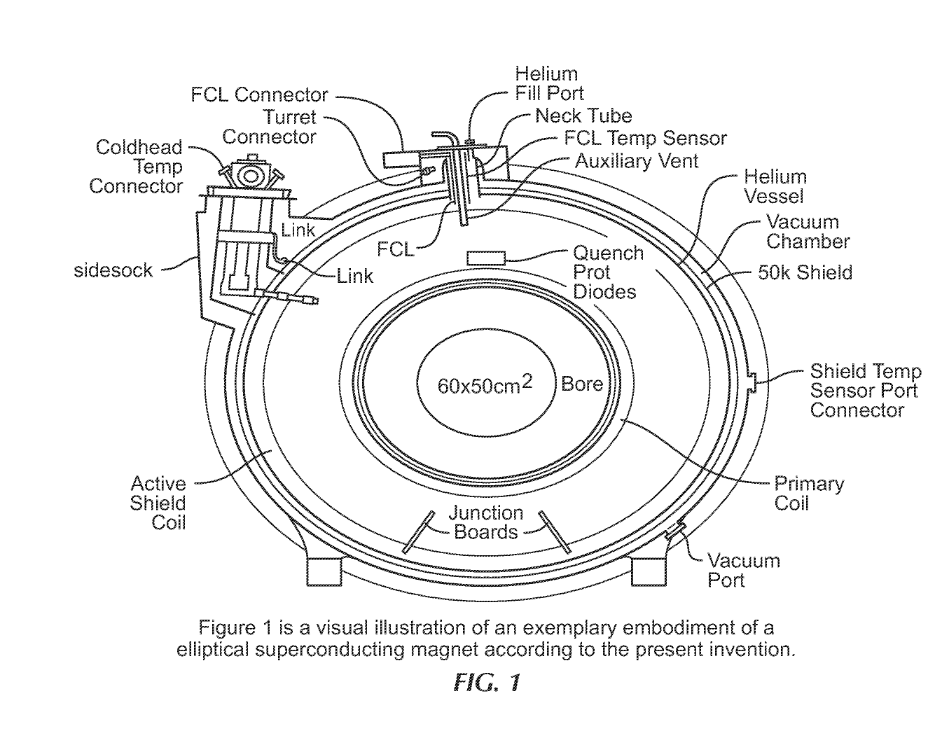

16. Apparatus for simultaneously acquiring magnetic resonance signals from two people in one magnetic resonance imaging scanner, comprising:
a. A elliptical cylinder inner bore superconducting or non-superconducting magnet that can generate homogeneous static magnetic field whose volume can accommodate two people, as shown in FIG. 1;
b. Three direction magnetic field gradient coils which can produce linear magnetic gradient region that can accommodate two people.
c. RF coils that can transmit and/or receive MR signals in the region that can accommodate two people.
d. Shimming coil set that can shim an elliptical volume that is large enough for accommodating two people.

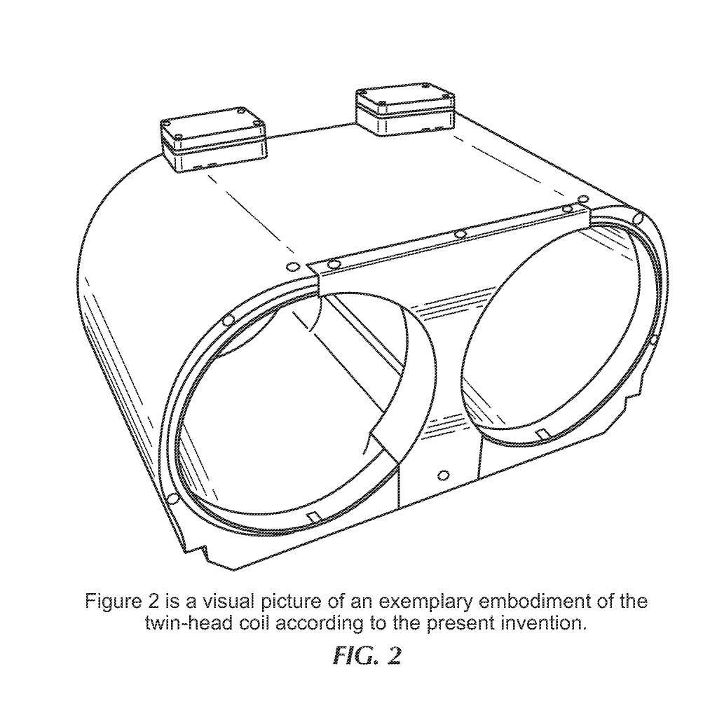

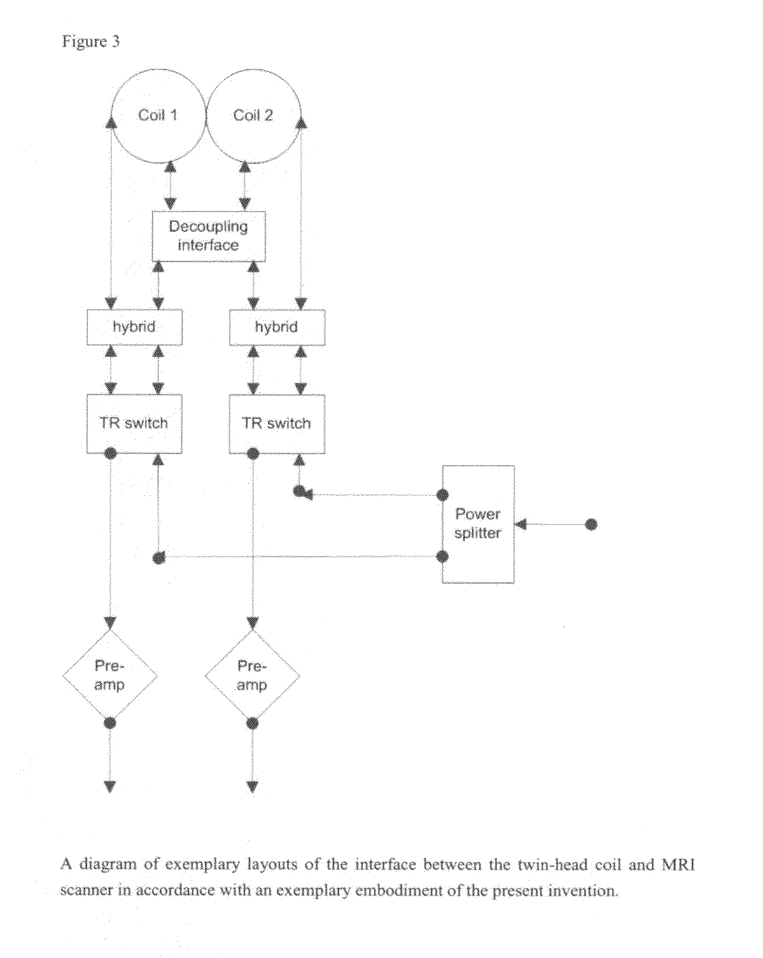

17. The apparatus of claim 16, wherein component c further includes a twin-head coil comprising:
a. Two circular polarized volume coils side-by-side for two people, as shown in FIG. 2.
b. The two component coils are decoupled by both their geometrical orientation and decoupling interface circuit system.
c. The interface between MRI scanner and the twin-head coil is shown in FIG. 3.

18. The apparatus of claim 17, wherein component b further includes a scheme for decoupling circular-polarized RF coils
